(19) United States
(12) Patent Application Publication
(10) Pub. No.: US 2006/0063827 A1
(43) Pub. Date: Mar. 23, 2006

(54) Systemic administration of therapeutic amino acids and N-acetylamino acids

(76) Inventors: Ruey J. Yu, Chalfont, PA (US); Eugene J. Van Scott, Abington, PA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/228,230

(22) Filed: Sep. 19, 2005

Related U.S. Application Data
(60) Provisional application No. 60/612,253, filed on Sep. 23, 2004.
(60) Provisional application No. 60/627,022, filed on Nov. 12, 2004.

(51) Int. Cl. A61K 31/401; A61K 31/198; A61K 31/366

(52) U.S. Cl. 514/423; 514/561; 514/460

(57) ABSTRACT

Embodiments relate to compositions and use of compositions comprising amino acids and/or N-acetylamino acids for systemic administration to a mammal. Systemic administration is believed to alleviate or improve symptoms or syndromes associated with nervous, vascular, musculoskeletal, or cutaneous systems.

This application claims priority under 35 U.S.C. §119 to Provisional Patent Application No. 60/612,253, filed on Sep. 23, 2004, and Provisional Patent Application No. 60/627,022, filed on Nov. 12, 2004, the disclosures of each of which are incorporated by reference herein in their entireties.

### FIELD OF THE INVENTION

Embodiments described herein relate to compositions and use of compositions comprising amino acids and/or N-acetylamino acids for systemic administration to a mammal. Systemic administration is believed to alleviate or improve symptoms or syndromes associated with nervous, vascular, musculoskeletal or cutaneous system.

### DESCRIPTION OF RELATED ART

PCT Application No. PCT/US96/16534, filed Oct. 16,1996, entitled “Topical Compositions Containing N-Acetylcysteine and Odor Masking Materials,” describes topical compositions comprising from 0.01% to 50% of N-acetylcysteine or a derivative of N-acetylcysteine, from 0.01% to 0.5% of an odor masking material, and a topical carrier to improve the appearance of skin. U.S. Pat. No. 5,132,113 entitled “Nutritional Composition Containing Essential Amino Acids” describes a nutritional composition comprising isoleucine, leucine, lysine, methionine, phenylalanine, threonine, tryptophan and valine in amounts that are said to provide a Net Nitrogen Utilization of at least 80%. U.S. Pat. No. 6,159,485 entitled “N-Acetyl Aldosamines, N-Acetylamino Acids and Related N-Acetyl Compounds and Their Topical Use”, U.S. Pat. No. 6,524,593 B1 entitled “N-Acetyl Aldosamines and Related N-Acetyl Compounds, and Their Topical Use,” and U.S. Pat. No. 6,808,716 B2 entitled “N-Acetylamino Acids, Related N-Acetyl Compounds and Their Topical Use,” describe and claim the use of compositions comprising N-acetylamino acids and N-acetyl aldosamines for topical treatment of cosmetic conditions and dermatological disorders. U.S. Pat. No. 6,824,786 B2 entitled “Compositions Comprising Phenyl-Glycine Derivatives” describes and claims the compositions and use of the compositions comprising phenyl-glycine derivatives for treating cosmetic conditions and dermatological disorders. The disclosures of each of the aforementioned United States patents are incorporated by reference herein in their entireties.

Compositions containing essential amino acids have been used orally as a dietary supplement for protein substitutes. Japanese Pat. No. 4,282,313 entitled “Antihypertensives Containing L-Arginine or its Salts” describes oral arginine for the treatment of hypertension.

Aspartic acid and asparagines have been taken orally for treatment of drug addiction, management of chronic fatigue and treatment of liver cirrhosis, as described in Clin. Nutr. 4 (Suppl.): 88-96, 1985 entitled “Administration of Aspartate to Patients with Liver Cirrhosis”; Bull. Narc. 35:11-15, 1983 entitled “The Treatment with L-Aspartic Acid of Persons Addicted to Opiates”; and Northwest Med. 60:597-603, 1961 entitled “Treatment of Fatigue with Aspartic Acid Salts”.

Cysteine and cystine have been used orally for treatment of acetaminophen poisoning as described in “Conn's Current Therapy”, pp 1099-1139, 1992, W. B. Saunders Comp. entitled “Acute Poisoning” and Ann. Rev. Pharmacol. Toxicol. 23:87-101, 1983 entitled “The Treatment of Acetaminophen Poisoning”.

Glutamic acid has been used orally for relief of mental retardation and epilepsy as described in “The Pharmacological Basis of Therapeutics” MacMillan Publishing Comp. 1985 entitled “Agents Affecting Volume and Composition of Body Fluids”.

Glutamine has been used orally for treatment of cystinuria and alcoholism as described in N. Engl. J. Med. 315:1120-1123, 1986 entitled “Anticystinuric Effects of Glutamine and of Dietary Sodium Restriction”; N. Engl. J. Med. 301:196-198, 1979 entitled “Effect of Glutamine on Cystine Excretion in a Patient with Cystinuria” and N. Engl. J. Med. 301:1066, 1979 entitled “Oral L-Glutamine Well Tolerated.”

Histidine has been used orally for treatment of rheumatoid arthritis as described in J. Rheumatol. 4:414-419, 1977 entitled “Treatment of Rheumatoid Arthritis with L-Histidine: A Randomized, Placebo-Controlled, Double-Blind Trial.”

Leucine has been used orally for treatment of Duchenne muscular dystrophy as described in Muscle Nerve 7:535-541, 1984 entitled “Clinical Investigation in Duchenne Muscular Dystrophy. IV. Double-Blind Controlled Trial of Leucine.”

Lysine has been used orally for treatment and prevention of herpes simplex lesions as described in Cutis 34:366-373, 1984 entitled “Treatment of Recurrent Herpes Simplex Infections with L-Lysine Monohydrochloride” and Arch. Dermatol. 121:167-168, 1985 entitled “Failure of Lysine in Frequently Recurrent Herpes Simplex Infection.”

Methionine has been used orally for improvement of inflammatory liver disease and treatment of acetaminophen poisoning as described in J. Am. Med. Assoc. 133:107, 1947 entitled “The Status of Methionine in the Prevention and Treatment of Liver Injury”; Ann. Rev. Pharmacol. Toxicol. 23:87-101, 1983 entitled “The Treatment of Acetaminophen Poisoning” and Arch. Intern. Med. 141:394-396, 1981 entitled “Treatment of Acetaminophen Poisoning.”

Phenylalanine has been used orally for treatment of pain, prevention or treatment of depression, treatment of hyperactivity, treatment of attention deficit disorder and mood changes and arousal as described in “Degradation of Endogenous Opioids: Its Relevance in Human Pathology and Therapy” vol. 5, pp. 207-215, 1983, Raven Press entitled “D-Phenylalanine in Human Chronic Pain”; Arch. Phys. Med. Rehabil. 67:436-439, 1986 entitled “Analgesic Effectiveness of D-Phenylalanine in Chronic Pain Patients”; “Nutrition and the Brain” Vol. 7, pp. 49-88, 1986 entitled “The Clinical Psychopharmacology of Tryptophan”; Am. J. Psychiatry 144:792-794, 1987 entitled “Treatment of Hyperactive Children with D-Phenylalanine”; Psychiatry Res. 16:21-26, 1985 entitled “Treatment of Attention Deficit Disorder with DL-Phenylalanine” and Physiol. Behav. 39:24-253, 1987 entitled “Phenylalanine and Aspartame Fail to Alter Feeding Behavior, Mood and Arousal in Men.”

Threonine has been used orally for modification of amyotrophic lateral sclerosis as described in Neurology 38 (Suppl.1):354-355, 1988 entitled “L-Threonine and the Modification of ALS.”

Tyrosine has been used orally for treatment of Parkinson's disease, attention deficit disorder, treatment of narcolepsy, treatment of hypertension, and treatment of depression as described in J. Am. Med. Assoc. 223:83, 1973 entitled “L-m-Tyrosine and Parkinsonism”; J. Am. Acad. Child Psychiatry 25:509-513, 1986 entitled “Amino Acid Supplementation as Therapy for Attention Deficit Disorder”; Lancet 2:1458-1459, 1988 entitled “Treatment of Narcolepsy with L-Tyrosine”; Am. J. Clin. Nutr. 38:429-435, 1983 entitled “The Influence of Oral Tyrosine and Tryptophan Feeding on Plasma Catecholamines in Man”; J. Affective Disord. 19:125-132, 1990 entitled “Tyrosine for Depression: A Double-Blind Trial” and Psychopharmacology 89:1-7, 1986 entitled “Evaluation of L-Tryptophan for Treatment of Insomnia: A Review.”

Tryptophan has been used orally for sleep aid, affective disorder and treatment of pain as described in Psychopharmacology 89:1-7, 1986 entitled “Evaluation of L-Tryptophan for Treatment of Insomnia: A Review”; Biol. Psychiatry 20:546-557, 1985 entitled “A Controlled Clinical Trial of L-Tryptophan in Acute Mania”; “Nutrition and the Brain” Vol. 7, pp. 89-138, 1986, Raven Press, entitled “Monoamine Precursors in the Treatment of Psychiatric Disorders”; “Nutrition and the Brain” Vol. 7, pp. 49-88, 1986, Raven Press, entitled “The Clinical Psychopharmacology of Tryptophan”; J. Neurosurg. 53:44-52, 1980 entitled “Pain and Tryptophan” and Pain 13:385-393, 1982 entitled “Alteration of Human Pain Thresholds by Nutritional Manipulation and L-Tryptophan Supplementation.”

Aspirin is commonly used for temporary relief of pain and inflammation of arthritis and bursitis. The most common side effect is a stomach irritation that may lead to gastrointestinal bleeding from long-term use. Corticosteroids such as prednisone and non-steroidal antiinflammatory drugs such as ibuprofen, naproxen, tolmetin and sulindac may also be used for temporary relief of arthritis. However, these drugs can also cause adverse side effects on long-term use.

The description herein of disadvantages and problems associated with known compositions, compounds, and methods is in no way intended to limit the scope of the embodiments described in this document to their exclusion. Indeed, certain embodiments may include one or more known composition, compound, or method without suffering from the so-noted disadvantages or problems.

### SUMMARY OF THE INVENTION

The inventors have discovered that certain amino acids and/or N-acetylamino acids by systemic administration are therapeutically effective for prevention or treatment to alleviate or improve symptoms or syndromes associated with the nervous, vascular, musculoskeletal or cutaneous systems. It therefore is a feature of an embodiment of the invention to provide a method of preventing or alleviating symptoms or syndromes associated with the nervous, vascular, muscoloskeletal, or cutaneous systems by sytemically administering to a patient or subject-in need thereof, a therapeutically effective amount of a composition comprising at least one amino acid selected from the group consisting of alanine, glycine, isoleucine, proline, serine, valine, β-alanine, γ-aminobutanoic acid, citrulline and ornithine, present as a free acid, salt, partial salt, amide, lactone, ester, anhydride, dimer, oligomer or polymer form, or present as stereoisomers such as D,L, or DL form, or non-stereoisomers such as glycine, or mixtures thereof.

In accordance with another feature of an embodiment, there is provided a method of preventing or alleviating symptoms or syndromes associated with the nervous, vascular, muscoloskeletal, or cutaneous systems by sytemically administering to a patient in need thereof, a therapeutically effective amount of a composition comprising at least one N-acetylamino acid selected from N-acetyl-proline or an N-acetyl aminoacid represented by the formula:
R₁CH(NHCOCH₃)(CH₂)ₙCOOR₂

Where R₁, R₂ is independently H, an alkyl, aralkyl or aryl group having 1 to 14 carbon atoms; n is an integer, preferably from 0 to 5; and in addition R₁ can carry OH, SH, SCH₃, NH₂, CONH₂, NHCONH₂, NHC(═NH)NH₂, imidazole, pyrrolidine or other heterocyclic group. The H attached to any carbon atom can be substituted by I, F, Cl, Br, OH or alkoxy group having 1 to 9 carbons. The N-acetylamino acids can be present as free acid, salt, partial salt, amide, ester, anhydride, lactone form, or stereoisomers such as D,L,or DL, or non-stereoisomers such as N-acetyl-glycine. Among commonly known N-acetylamino acids, N-acetyl-proline cannot be represented by the above generic structure because the alpha amino group is part of the heterocyclic pyrrolidine ring.

These and other features of various embodiments will become readily apparent to those skilled in the art upon review of the detailed description that follows.

### DESCRIPTION OF THE PREFERRED EMBODIMENTS

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth.

Certain amino acids and/or N-acetylamino acids by systemic administration are therapeutically effective or beneficial for disorders associated with the nervous, vascular, musculoskeletal, or cutaneous systems. For example, subjects or patients with eczema and itchy skin were relieved from the itch and the improvement of eczema lesions by oral administration of L-proline 2-3 g twice daily. The itch usually disappeared within 24 hours and the eczema lesions improved within a few days after oral administration of the amino acid. Subjects or patients having early to progressive signs or symptoms of Alzheimer's disease as evidenced by short and medium term loss of memory improved cognitive knowledge after oral administration of N-acetyl-L-glutamic acid or N-acetyl-L-glutamine.

Throughout this description, the symptoms and syndromes associated with the nervous system include (1) Alzheimer's disease: progressive loss of memory, shrinkage and atrophy of cerebral cortex, tangles of fibers in nerve cells, senile plaques of amyloid, decreased choline acetyltransferase enzyme, (2) Carpal tunnel syndrome: weakness, pain, tingling, numbness, burning in palm and fingers, (3) Encephalitis: inflammation of the brain, (4) Headache: migraine, expansion of blood vessels pressing on nerves or constriction blocking blood supply, inflammation, muscle contraction to face, neck or scalp, (5) Meningitis: infection of spinal fluid and meninges, (6) Neuralgia: nerve pain, peripheral neuropathy, sciatica, shingles, trigeminal neuralgia, (7) Parkinson's disease: tremors in limbs, muscular rigidity, (8) Amnesia: loss of memory and inability to form new memory, and others such as ataxia, Bell's palsy, epilepsy, multiple sclerosis, myasthenia gravis, narcolepsy, paralysis and rabies.

Throughout this description, vascular conditions, reactions and disorders include acanthosis nigricans, acrocyanosis, actinic cheilitis, actinic prurigo, dermatitis, dermatosis, dermographism, dyshidrosis, drug eruptions, eczema, erythema, erythema migrans, erythrocyanosis, erythromelalgia, familial hemorrhage, histamine reaction, inflammatory papular and pustular lesions, lichen planus, lupus erythematosus, mycosis fungoides, neurodermatitis, neuropeptide and neurovascular reactions, parapsoriasis, perniosis(chilblains), photoallergy, photoreaction, photosensitivity, pityriasis rosea, pityriasis rubra pilaris, polymorphic light eruption, psoriasis, rhinophyma, rosacea, sclerosis, spider naevi, T-cell disorders, telangiectasia, urticaria and other vascular reactions.

The abnormalities of musculoskeletal system include (1) Osteoporosis: reduction of calcium in bone leading to thin and susceptible to fracture, (2) Osteoarthritis: inflammation of joint cartilage provoking swelling and pain, (3) Rheumatoid arthritis: inflammation of synovium and destructions of cartilage, damage to heart, lungs, nerves and eyes, (4) Ankylosing spondylitis: arthritis affecting sacroiliac joints and spine with inflammation and immovability, (5) Bursitis: inflammation of bursa, (6) Tendinitis: inflammation of tendon, (7) Gout: recurrent acute arthritis from uric acid deposit, and others such as backache, bunion and hernia.

The disorders or abnormal cutaneous system include disturbed keratinization, pigmentation and immunity; inflammation; infections and decreased physiological functions. The indications include acne, itch, eczema, psoriasis, signs of aging, changes or damage to skin, nail and hair associated with intrinsic aging and/or extrinsic aging, as well as changes or damage caused by extrinsic factors such as sunlight, air pollution, wind, cold, heat, dampness, chemicals, smoke, cigarette smoking, radiations including electromagnetic radiations and ionizing radiations.

The systemic administration includes injection, infusion, oral and other route; the preferred one is by oral administration. Oral administration of certain amino acids and/or N-acetylamino acids is beneficial and can improve cognition and memory performance in Alzheimer's subjects, knee joints of osteoarthritis, and cutaneous system including deranged or disordered cutaneous tissues. The manifestations of cutaneous disorders can include acne; age spots; blemished skin; blotches; cellulite; dermatoses; dandruff; dry skin; pruritus, eczema; ichthyosis; keratoses and hyperkeratoses; lentigines; melasmas; mottled skin; pseudofolliculitis barbae; photoaging and photodamage; psoriasis; skin lines; stretch marks; thinning of skin, nail plate and hair; wrinkles; xerosis; oral or gum disease; irritated, inflamed, unhealthy, damaged or abnormal mucosa, skin, hair, nail, nostril, ear canal, anal or vaginal conditions; defective synthesis or repair of dermal components; abnormal or diminished synthesis of collagen, glycosaminoglycans, proteoglycans and elastin as well as diminished levels of such components in the dermis; uneven and rough surface of skin, nail and hair; loss or reduction of skin, nail and hair resiliency, elasticity and recoilability; lack of skin, nail and hair lubricants and luster; fragility and splitting of nail and hair; yellowing skin; reactive, irritating or telangiectatic skin; dull and older-looking skin, nail and hair; for skin bleach and lightening and wound healing.

The compounds of the embodiments can be divided into the following groups: (a) certain amino acids; and (b) certain N-acetylamino acids, and combinations and derivatives thereof.

(A) The amino acid useful in the embodiments preferably is selected from the group consisting of alanine, glycine, isoleucine, proline, serine, valine, β-alanine, γ-aminobutanoic acid, citrulline and ornithine as free acid, salt, partial salt, amide, lactone, ester, anhydride, dimer, oligomer or polymer form, and can be present as stereoisomers such as D,L, or DL form, or non-stereoisomers such as glycine.

As an illustration, proline and its derivatives can be represented as follows:

(1) L-proline, sodium L-prolinate, L-prolinamide, ethyl L-prolinate, methyl L-prolinate, propyl L-prolinate, L-Pro-L-Pro dimer, (L-Pro-)₈ oligomer, ((L-Pro-)₂₀ polymer,

(2) D-proline, sodium D-prolinate, D-prolinamide, ethyl D-prolinate, methyl D-prolinate, propyl D-prolinate

(3) DL-proline, sodium DL-prolinate, DL-prolinamide, ethyl DL-prolinate, methyl DL-prolinate, propyl DL-prolinate

(B) The N-acetylamino acids useful in the embodiment preferably may be represented by the following generic structure:
R₁CH(NHCOCH₃)(CH₂)ₙCOOR₂

Where R₁, R₂ is independently H, an alkyl, aralkyl or aryl group having 1 to 14 carbon atoms; n is an integer, preferably from 0 to 5; and in addition R₁ can carry OH, SH, SCH₃, NH₂, CONH₂, NHCONH₂, NHC(═NH)NH₂, imidazole, pyrrolidine or other heterocyclic group. The H attached to any carbon atom can be substituted by I, F, Cl, Br, OH or alkoxy group having 1 to 9 carbons. The N-acetylamino acids can be present as free acid, salt, partial salt, amide, ester, anhydride, lactone form, or stereoisomers such as D,L, or DL, or non-stereoisomers such as N-acetyl-glycine. Among commonly known N-acetylamino acids, N-acetyl-proline cannot be represented by the above generic structure because the alpha amino group is part of the heterocyclic pyrrolidine ring.

Representative N-acetylamino acids can be selected from N-acetyl-alanine, N-acetyl-β-alanine, N-acetyl-γ-aminobutanoic acid, N-acetyl-β-aminoisobutanoic acid, N-acetyl-arginine, N-acetyl-asparagine, N-acetyl-aspartic acid, N-acetyl-citrulline, N-acetyl-dopa (N-acetyl-3,4-dihydroxyphenylalanine), N-acetyl-glycine, N-acetyl-glutamic acid, N-acetyl-glutamine, N-acetyl-histidine, N-acetyl-homoserine, N-acetyl-4-hydroxyproline, N-acetyl-isoleucine, N-acetyl-leucine, N-acetyl-lysine, N-acetyl-methionine, N-acetyl-ornithine, N-acetyl-phenylalanine, N-acetyl-proline, N-acetyl-serine, N-acetyl-threonine, N-acetyl-tryptophan, N-acetyl-tyrosine and N-acetyl-valine.

As an illustration, N-acetyl-proline and its derivatives can be represented as follows:

(1) N-acetyl-L-proline, sodium N-acetyl-L-prolinate, N-acetyl-L-prolinamide, ethyl N-acetyl-L-prolinate, methyl N-acetyl-L-prolinate, propyl N-acetyl-L-prolinate

(2) N-acetyl-D-proline, sodium N-acetyl-D-prolinate, N-acetyl-D-prolinamide, ethyl N-acetyl-D-prolinate, methyl N-acetyl-D-prolinate, propyl N-acetyl-D-prolinate

(3) N-acetyl-DL-proline, sodium N-acetyl-DL-prolinate, N-acetyl-DL-prolinamide, ethyl N-acetyl-DL-prolinate, methyl N-acetyl-DL-prolinate, propyl N-acetyl-DL-prolinate

For synergetic or synergistic therapeutic effects, vitamins, cosmetics, pharmaceutical and/or other agents can be used topically or taken systemically at the same time or sequentially to complement or enhance therapeutic effects of the amino acids or N-acetylamino acids. Examples of the above agents include abacavir, acebutolol, acetaminophen, acetaminosalol, acetazolamide, acetohydroxamic acid, acetylsalicylic acid, acitretin, aclovate, acrivastine, actiq, acyclovir, adapalene, adefovir dipivoxil, adenosine, albuterol, alfuzosin, allopurinol, alloxanthine, almotriptan, alprazolam, alprenolol, aluminum acetate, aluminum chloride, aluminum chlorohydroxide, aluminum hydroxide, amantadine, amiloride, aminacrine, aminobenzoic acid (PABA), aminocaproic acid, aminosalicylic acid, amiodarone, amitriptyline, amlodipine, amocarzine, amodiaquin, amorolfine, amoxapine, amphetamine, ampicillin, anagrelide, anastrozole, anthralin, apomorphine, aprepitant, arbutin, aripiprazole, ascorbic acid, ascorbyl palmitate, atazanavir, atenolol, atomoxetine, atropine, azathioprine, azelaic acid, azelastine, azithromycin, bacitracin, beclomethasone dipropionate, bemegride, benazepril, bendroflumethiazide, benzocaine, benzonatate, benzophenone, benzoyl peroxide, benztropine, bepridil, betamethasone dipropionate, betamethasone valerate, brimonidine, brompheniramine, bupivacaine, buprenorphine, bupropion, burimamide, butenafine, butoconazole, cabergoline, caffeic acid, caffeine, calcipotriene, camphor, candesartan cilexetil, capsaicin, carbamazepine, carbamide peroxide, cefditoren pivoxil, cefepime, cefpodoxime proxetil, celecoxib, cetirizine, cevimeline, chitosan, chlordiazepoxide, chlorhexidine, chloroquine, chlorothiazide, chloroxylenol, chlorpheniramine, chlorpromazine, chlorpropamide, ciclopirox, cilostazol, cimetidine, cinacalcet, ciprofloxacin, citalopram, citric acid, cladribine, clarithromycin, clemastine, clindamycin, clioquinol, clobetasol propionate, clomiphene, clonidine, clopidogrel, clotrimazole, clozapine, coal tar, cocaine, codeine, cromolyn, crotamiton, cyclizine, cyclobenzaprine, cycloserine, cytarabine, dacarbazine, dalfopristin, dapsone, daptomycin, daunorubicin, deferoxamine, dehydroepiandrosterone, delavirdine, desipramine, desloratadine, desmopressin, desoximetasone, dexamethasone, dexmedetomidine, dexmethylphenidate, dexrazoxane, dextroamphetamine, diazepam, dicyclomine, didanosine, dihydrocodeine, dihydromorphine, diltiazem, 6,8-dimercaptooctanoic acid (dihydrolipoic acid), diphenhydramine, diphenoxylate, dipyridamole, disopyramide, dobutamine, dofetilide, dolasetron, donepezil, dopa esters, dopamide, dopamine, dorzolamide, doxepin, doxorubicin, doxycycline, doxylamine, doxypin, duloxetine, dyclonine, econazole, eflornithine, eletriptan, emtricitabine, enalapril, ephedrine, epinephrine, epinine, epirubicin, eptifibatide, ergotamine, erythromycin, escitalopram, esmolol, esomeprazole, estazolam, estradiol, ethacrynic acid, ethinyl estradiol, etidocaine, etomidate, famciclovir, famotidine, felodipine, fentanyl, ferulic acid, fexofenadine, flecainide, fluconazole, flucytosine, fluocinolone acetonide, fluocinonide, 5-fluorouracil, fluoxetine, fluphenazine, flurazepam, fluvoxamine, formoterol, furosemide, galactarolactone, galactonic acid, galactonolactone, galantamine, gatifloxacin, gefitinib, gemcitabine, gemifloxacin, glucarolactone, gluconic acid, gluconolactone, glucuronic acid, glucuronolactone, glycolic acid, griseofulvin, guaifenesin, guanethidine, N-guanylhistamine, haloperidol, haloprogin, hexylresorcinol, homatropine, homosalate, hydralazine, hydrochlorothiazide, hydrocortisone, hydrocortisone 21-acetate, hydrocortisone 17-butyrate, hydrocortisone 17-valerate, hydrogen peroxide, hydromorphone, hydroquinone, hydroquinone monoether, hydroxyzine, hyoscyamine, hypoxanthine, ibuprofen, ichthammol, idarubicin, imatinib, imipramine, imiquimod, indinavir, indomethacin, irbesartan, irinotecan, isoetharine, isoproterenol, itraconazole, kanamycin, ketamine, ketanserin, ketoconazole, ketoprofen, ketotifen, kojic acid, labetalol, lactic acid, lactobionic acid, lamivudine, lamotrigine, lansoprazole, letrozole, leuprolide, levalbuterol, levofloxacin, lidocaine, linezolid, lobeline, loperamide, losartan, loxapine, lysergic diethylamide, mafenide, malic acid, maltobionic acid, mandelic acid, maprotiline, mebendazole, mecamylamine, meclizine, meclocycline, memantine, menthol, meperidine, mepivacaine, mercaptopurine, mescaline, metanephrine, metaproterenol, metaraminol, mefformin, methadone, methamphetamine, methotrexate, methoxamine, methyldopa esters, methyldopamide, 3,4-methylenedioxymethamphetamine, methyllactic acid, methyl nicotinate, methylphenidate, methyl salicylate, metiamide, metolazone, metoprolol, metronidazole, mexiletine, miconazole, midazolam, midodrine, miglustat, minocycline, minoxidil, mirtazapine, mitoxantrone, moexiprilat, molindone, monobenzone, morphine, moxifloxacin, moxonidine, mupirocin, nadolol, naftifine, nalbuphine, nalmefene, naloxone, naproxen, nefazodone, nelfinavir, neomycin, nevirapine, nicardipine, nicotine, nifedipine, nimodipine, nisoldipine, nizatidine, norepinephrine, nystatin, octopamine, octreotide, octyl methoxycinnamate, octyl salicylate, ofloxacin, olanzapine, olmesartan medoxomil, olopatadine, omeprazole, ondansetron, oxiconazole, oxotremorine, oxybenzone, oxybutynin, oxycodone, oxymetazoline, padimate O, palonosetron, pantothenic acid, pantoyl lactone, paroxetine, pemoline, penciclovir, penicillamine, penicillins, pentazocine, pentobarbital, pentostatin, pentoxifylline, pergolide, perindopril, permethrin, phencyclidine, phenelzine, pheniramine, phenmetrazine, phenobarbital, phenol, phenoxybenzamine, phentolamine, phenylephrine, phenylpropanolamine, phenytoin, physostigmine, pilocarpine, pimozide, pindolol, pioglitazone, pipamazine, piperonyl butoxide, pirenzepine, podofilox, podophyllin, povidone iodine, pramipexole, pramoxine, prazosin, prednisone, prenalterol, prilocaine, procainamide, procaine, procarbazine, promazine, promethazine, promethazine propionate, propafenone, propoxyphene, propranolol, propylthiouracil, protriptyline, pseudoephedrine, pyrethrin, pyrilamine, pyrimethamine, quetiapine, quinapril, quinethazone, quinidine, quinupristin, rabeprazole, reserpine, resorcinol, retinal, 13-cis retinoic acid, retinoic acid, retinol, retinyl acetate, retinyl palmitate, ribavirin, ribonic acid, ribonolactone, rifampin, rifapentine, rifaximin, riluzole, rimantadine, risedronic acid, risperidone, ritodrine, rivastigmine, rizatriptan, ropinirole, ropivacaine, salicylamide, salicylic acid, salmeterol, scopolamine, selegiline, selenium sulfide, serotonin, sertindole, sertraline, shale tar, sibutramine, sildenafil, sotalol, streptomycin, strychnine, sulconazole, sulfabenz, sulfabenzamide, sulfabromomethazine, sulfacetamide, sulfachlorpyridazine, sulfacytine, sulfadiazine, sulfadimethoxine, sulfadoxine, sulfaguanole, sulfalene, sulfamethizole, sulfamethoxazole, sulfanilamide, sulfapyrazine, sulfapyridine, sulfasalazine, sulfasomizole, sulfathiazole, sulfisoxazole, sulfur, tadalafil, tamsulosin, tartaric acid, tazarotene, tegaserol, telithromycin, telmisartan, temozolomide, tenofovir disoproxil, terazosin, terbinafine, terbutaline, terconazole, terfenadine, tetracaine, tetracycline, tetrahydrozoline, theobromine, theophylline, thiabendazole, thioctic acid (lipoic acid), thioridazine, thiothixene, thymol, tiagabine, timolol, tinidazole, tioconazole, tirofiban, tizanidine, tobramycin, tocainide, tolazoline, tolbutamide, tolnaftate, tolterodine, tramadol, tranylcypromine, trazodone, triamcinolone acetonide, triamcinolone diacetate, triamcinolone hexacetonide, triamterene, triazolam, triclosan, triflupromazine, trimethoprim, trimipramine, tripelennamine, triprolidine, tromethamine, tropic acid, tyramine, undecylenic acid, urea, urocanic acid, ursodiol, vardenafil, venlafaxine, verapamil, vitamin E acetate, voriconazole, warfarin, wood tar, xanthine, zafirlukast, zaleplon, zinc pyrithione, ziprasidone, zolmitriptan, zolpidem

Systemic administration, or systemically administration, as those phrases are utilized herein, denotes administration of a composition into the body via oral administration or parenteral injection. The amino acids and/or N-acetylamino acids useful in the embodiments may be formulated for oral administration or for parenteral injections. In oral preparations, the amino acids and/or N-acetylamino acids can be formulated in tablet form or in gelatin capsules with or without mixing with gelatin powder. Each tablet or capsule can contain from 20 to 500 mg of the amino acids and/or N-acetylamino acids. For parenteral injections, the amino acids and/or N-acetylamino acids are prepared under sterilized conditions usually in 1 to 10% concentration dissolved in, or in fine suspension in water, propylene glycol, sesame oil or other non-aqueous vehicle.

The amino acids and N-acetylamino acids useful in the embodiments can be used as free acid, amide, ester, lactone, anhydride, salt or partial salt form. The route of systemic administration can be by injection, infusion, oral, or the like. For simplicity and convenience oral administration is preferred. The amino acids and N-acetylamino acids of the present invention in capsule, tablet or powder form can be orally taken by subjects or patients a few times daily with preferred frequency of once or twice daily. The powder form can be taken directly with a spoon and followed by water or by dissolving the powder in fruit juice first. It is preferred that the composition is in the form of a powder, solution, suspension, juice, tablet or gelatin capsule, and that the amino acid is present in the composition in an amount with the range of from about 0.1 to 5 g.

In general, the powder of most amino acids in a level teaspoon weighs approximately between 2.0 to 3.0 g. The oral dosage for the amino acid can be from one to ten grams daily with preferred dosage of from 4 to 5 g daily, preferably divided into two equal amounts taken orally twice daily. The oral dosage for the N-acetylamino acid can be from 10 to 500 mg daily with preferred dosage of from 30 to 300 mg daily, preferably divided into two equal amounts taken orally twice daily.

As an example, a subject or patient having cutaneous disorders such as itchy eczema was instructed to take twice daily oral L-proline powder 2 g with or without in fruit juice, in a total amount of 4 g daily. The itch usually disappeared or diminished within 24 hours after taking oral L-proline powder.

On continued oral administration of L-proline, eczema lesions usually improved or completely ameliorated. The remission time varied depending on the patients and the severity of the disease. As long as the oral L-proline was continued, the skin of most patients remained free of the disease.

Oral administration of L-proline or glycine in normal human subject with dosage of 1 to 4 g also was found to provide calming or tranquilizing effect. Embodiments therefore encompass systemic administration of compositions comprising L-proline or glycine, or mixtures thereof, where the amount of L-proline or glycine is within the range of from about 0.5 to about 10 g, preferably from about 1 to about 4 grams.

Subjects or patients having symptoms or syndromes of nervous, vascular, musculoskeletal or cutaneous system were instructed to take oral amino acid or N-acetylamino acid on a daily basis. For subjective disorders such as pain, itch or the like the therapeutic effects were evaluated or judged by the subjects or patients; for example, if the pain or itch had disappeared within hours or days. For other detectable symptoms or syndromes, the therapeutic effects or improvements were evaluated or judged by medical professionals.

The particularly preferred amino acids useful in the embodiments described herein, and that can be administered systemically include alanine, glycine, isoleucine, proline, serine, valine, β-alanine, γ-aminobutanoic acid, citrulline and ornithine as free acid, salt, partial salt, amide, ester, anhydride or lactone form.

The particularly preferred N-acetylamino acids useful in the embodiments described herein, and that can be administered systemically include N-acetyl-alanine, N-acetyl-β-alanine, N-acetyl-γ-aminobutanoic acid, N-acetyl-β-aminoisobutanoic acid, N-acetyl-arginine, N-acetyl-asparagine, N-acetyl-aspartic acid, N-acetyl-citrulline, N-acetyl-dopa (N-acetyl-3,4-dihydroxyphenylalanine), N-acetyl-glycine, N-acetyl-glutamic acid, N-acetyl-glutamine, N-acetyl-histidine, N-acetyl-homoserine, N-acetyl-4-hydroxyproline, N-acetyl-isoleucine, N-acetyl-leucine, N-acetyl-lysine, N-acetyl-methionine, N-acetyl-ornithine, N-acetyl-phenylalanine, N-acetyl-proline, N-acetyl-serine, N-acetyl-threonine, N-acetyl-tryptophan, N-acetyl-tyrosine and N-acetyl-valine.

The following are illustrative treatment procedures. A representative protocol is described herein for treating Alzheimer's Disease with N-acetylamino acids. A subject or patient having Alzheimer's Disease with short-term loss of memory usually takes various vitamins and medications to slow down the progress of the disease. There are no pharmaceutical drugs presently available, however, that can reverse or even stop the progression of the disease. Typically administered vitamins and drugs such as donepezil, memantine, melatonin, lipoic acid, selenium and folic acid, have minimal benefits with a slow progression of mental deterioration, and little improvement in quality of life. The patient's memory usually is gone and recognition of family members and caregivers is minimal. The patient sleeps long hours such as more than 15 hours daily. Episodes of urinary and bowel incontinence occur daily.

An N-Acetylamino acid such as N-Acetyl-glutamic acid can be initiated at a low dose, which then can be gradually increased, for example, according to the following schedule. N-Acetyl-glutamic acid 4 grams may be dissolved in 40% ethanol (vodka) providing a 4% solution. Initially, N-acetyl-glutamic acid (20 mg in 0.5 ml vodka) can be added to fruit juice for convenience of oral administration. This dose of 20 mg preferably can be administered twice daily for 2 weeks, at which time the dose can be increased to 40 mg twice daily for 3 weeks. Within a several month treatment period, the patient will show signs of regaining short-term memory for events of the day. Recognition of family members and caregivers can improve, as well as calling them by name. Physical activity also may be increased. Sense of humor can appear, and the patient attempts to initiate a verbal conversation. Situational recognition can return to a substantial degree.

Preferred dosage ranges from 20 to 100 mg daily. The inventors have discovered that N-acetylamino acids such as N-acetyl-glutamic acid can cause distinct improvement in multiple signs that gauge severity status of Alzheimer's Disease. Using the guidelines provided herein, a person skilled in the art would be capable of determining the optimal dosage, depending on the disease progression, and physical characteristics of the patient in need.

The present inventors also have discovered that most vitamins, cosmetic and pharmaceutical agents can have synergetic or synergistic effects when used in combination with the amino acids and N-acetylamino acids described in the embodiments. These agents can be used or taken simultaneously or sequentially with oral administration of the amino acids or N-acetylamino acids to provide complementary or enhancing effects.

Particularly preferred embodiments now will be-described in more detail with reference to the following non-limiting examples.

### EXAMPLE 1

Gelatin capsules containing N-acetylamino acid or N-acetylamino amide with or without gelatin powder for oral administration were prepared as follows:

Lilly empty gelatin capsules were filled with fine crystalline powders of N-acetyl-L-proline or N-acetyl-L-prolinamide with or without gelatin powder.

Each capsule thus prepared contained approximately 25, 50, 100, 150 or 200 mg of N-acetyl-L-proline or N-acetyl-L-prolinamide. Under the same conditions, N-acetyl glutamic acid was prepared as 25, 50, 100 and 150 mg capsules.

### EXAMPLE 2

A female subject, age 76, had early signs of Alzheimer's disease as shown by loss of short term memory, inability of performing routine tasks such as cooking, washing etc. and failure of recognizing family members and names. Oral administration of N-acetyl-L-glutamic acid 100 mg capsule two times daily for three weeks improved her condition substantially so that she could now recognize the family members and recall certain events that had happened recently.

### EXAMPLE 3

A male subject, age 71, had acute eruption of nummular eczema with intense itch over the body. Oral medications such as prednisone and diphenhydramine gave limited relief of the itch. Oral administration of N-acetyl-L-prolinamide 100 mg four times daily for two days eradicated itch completely and improved eczema lesions substantially. The clinical improvement of the eczema lesions had been judged to be 75% improved after two days of oral administration. This result shows that systemic administration of N-acetyl-L-prolinamide is therapeutically effective for symptoms or syndromes associated with nervous and/or cutaneous systems.

### EXAMPLE 4

A female subject, age 65, with chronic patchy eczema of legs and trunk for 5 years, had itching and eczema partially controlled with topical medications including corticosteroids. The subject was administered orally L-proline powder dissolved in fruit juice, 1 level teaspoon (2.5 g) twice daily. Itching stopped within 3-4 hours. Eczema disappeared within 7-10 days. The subject has been free of eczema for 3 months. Whenever an area begins to itch the subject takes orally 1 teaspoon of L-proline and itching disappears within 2 hours. Eczema and itch are controlled with L-proline taken orally as necessary to eradicate itching.

### EXAMPLE 5

A female subject, age 91, had patchy eczema of the lower legs for 10 years. Itching and skin eruption were substantially relieved with topical use of corticosteroids and other medications, e.g. topical 2% diphenhydramine and topical 5% N-acetyl proline ethyl ester. Upon beginning oral L-proline, 1 level teaspoon (2.5 g) twice daily, the subject noted that itching promptly disappeared within hours and has remained so for one month. The subject now takes ½ teaspoon of L-proline orally twice daily with complete control of itching, and continual resolution of patchy eczema.

### EXAMPLE 6

A female subject, age 85, with large plaques of eczema on the forearms and upper arms for 4 years. Substantial but incomplete control was achieved with topical medications. Upon initiating oral L-proline, 1 level teaspoon (2.5 g) twice daily itching promptly was relieved. With sustained use of oral L-proline, appearance of skin slowly is returning toward normal.

### EXAMPLE 7

A female subject, age 92, with chronic patchy eczema for 12 years duration did not respond to topical treatment with corticosteroids. A combination of topical corticosteroid formulations and oral antihistamines provided some relief of symptoms but not eczematous lesions on her legs. The subject was administered orally 2 g L-proline powder twice daily in water or fruit juice. The itch associated with eczema lesions disappeared within one hour after oral administration of L-proline powder. Daily intake of L-proline provided sustained relief of itch and improvement of eczema lesions. After two weeks of such treatment, pruritus disappeared completely and eczema lesions improved almost completely.

### EXAMPLE 8

A male subject, age 87, with chronic large erythematous plaques, 1 to 8 inches in diameter, of intensively pruritic eczema and with generalized pruritus, did not respond to conventional therapies including topical corticosteroids, oral prednisone and antihistamines for the past two years. The subject took orally L-proline powder 2 g twice daily and generalized pruritus began to disappear within one week. After three weeks of oral administration, all symptoms of pruritus had disappeared completely. Concomitant topical treatment with corticosteroid formulations and emollients plus oral L-proline resulted in almost complete disappearance of clinical signs of eczematous plaques.

### EXAMPLE 9

A male subject, age 72, with chronic itchy eczema took oral L-proline 2 g twice daily (total 4 g per day) for a week. The itch of the skin disappeared during the second day of oral administration. The eczema lesions were also improved after one week of oral administration of L-proline. Oral administration of L-proline at dosage of 1-2 g has been found to have a tranquilizing effect without drowsiness.

### EXAMPLE 10

A female subject, age 65, with patchy eczema involving entire body of approximately 10 years duration had intense pruritus (itch). Over a period of 14 months she took L-proline orally, 2 grams powder dissolved in fruit juice twice daily. Such treatment provided complete relief of itch within 24 hours. Clinical lesions of eczema cleared completely within one month of oral administration. Without oral administration of L-proline, eczema recurred after 5 months. Resumption of oral L-proline provided complete relief of itch within two weeks although visible signs of eczema dermatitis remained. However, with sustained oral intake of L-proline, pruritus and visible signs of eczema disappeared after 2-3 months and had remained so with daily intake of L-proline 2 grams daily.

### EXAMPLE 11

A male subject, age 88, had patchy eczema with pruritus for 2 years duration. Treatment included topical medications, and L-proline powder orally 2.5 grams twice daily. Complete resolution of clinical signs of eczema and complete disappearance of pruritus occurred over a two-month interval. Topical therapy was then discontinued and daily dosage of L-proline was maintained for 2 weeks and then discontinued. Three weeks after discontinuation of oral L-proline, pruritus recurred. Pruritus had variously been controlled, from completely to incompletely with subsequent oral intake of L-proline.

### EXAMPLE 12

A male subject, age 72, having itchy eczema took oral glycine powder 2 g twice daily for a total of 4 g per day. At the end of second day, the itch diminished substantially. Oral administration of glycine at dosage of 4 g per day had been found to have a tranquilizing effect without drowsiness. This result shows that oral administration of glycine is therapeutically effective for symptoms or syndromes associated with nervous and cutaneous systems.

### EXAMPLE 13

A female subject, age 92, had patchy eczema and intense pruritus for 13 years duration. The intense pruritus was not controlled by topical corticosteroids, all generally available topical anti-itch products or by oral antihistamines. Oral administration of L-proline powder, 2 grams twice daily, completely relieved pruritus within 2 days and with sustained dosing has been associated with complete remission of all signs and symptoms of eczema. Discontinuation of L-proline intake is followed by recurrence of eczema within 1-2 weeks.

### EXAMPLE 14

A female subject, age 39, having patchy eczema and pruritus since age 16, was somewhat improved by topical and oral corticosteroids. Distinct improvement of skin lesions, and of itch had been achieved with topical formulations containing N-acetyl-proline ethyl ester 7% concentration. Addition of oral L-proline 2 grams, twice daily, provided substantial additional improvement of itch, which was also associated with clearing of skin lesions.

### EXAMPLE 15

Several amino acids can be orally administered concomitantly or sequentially as shown in the following example. A male subject, age 72, having chronic itchy eczema on both hands took concomitantly oral glycine powder 2 g and L-proline powder 2 g once daily for 7 days. The itch on both hands disappeared after 7 days of oral administration. The same dosages were repeated for the next 7 days except that glycine was taken orally in the morning and L-proline was taken orally in the afternoon. At the end of 14 days, the eczema lesions improved substantially. This result shows that oral administration of combination amino acids can provide therapeutic effects for symptoms or syndromes associated with nervous and cutaneous systems.

### EXAMPLE 16

Several different amino acids can be orally administered concomitantly or sequentially. A male subject, age 73, having atopic itchy eczema on both hands took concomitantly oral L-proline powder 1 g and L-arginine powder 1.5 g twice daily for three days. The itch on both hands disappeared after 3 days of oral administration. The eczema lesions improved substantially. This result shows that oral administration of combination amino acids can provide therapeutic effects for symptoms or syndromes associated with nervous and cutaneous systems

### EXAMPLE 17

A female subject, age 70, had 2 plaques of eczema on skin of left upper back, present for 5-6 years, unresponsive to generally available topical therapy. Lesions were pruritic and interfered with sleeping. Upon her taking L-proline orally, 2 grams twice daily, itching disappeared completely within 24 hours. Oral L-proline was continued at that dosage and 6 weeks later lesions had cleared, and pruritus was completely relieved.

### EXAMPLE 18

A female subject, age 87, had diffuse eczema of the arms for 11-12 years.

Topical corticosteroid therapy provided minimal relief of itching and minimal improvement of skin lesions. Oral intake of L-proline 2 grams twice daily, over the course of 5 months, had provided complete relief of itch and return of the skin to normal appearance.

### EXAMPLE 19

Oral administration of N-acetylamino acids for treatment of nervous disorders including Alzheimer's disease can be described as follows. A female subject, age 79, with Alzheimer's Disease of 13 years duration. Treatment during the first 12 years included donepezil and memantine; antioxidants Vitamin E, Vitamin C, citric acid, tartaric acid, lipoic acid, selenium; multivitamin tablets; Vitamin B6 with Vitamin B12 and folic acid and melatonin. The foregoing treatments were associated with a slow rate of progression of mental deterioration, but after 12 years all memory was gone and recognition of family members and caregivers was minimal. The patient would sleep 15-18 hours daily. Episodes of urinary and bowel incontinence occurred daily.

N-Acetyl-glutamic acid treatment was initiated at a low dose, which was gradually increased according to the following schedule. N-Acetyl-glutamic acid 4 g was dissolved in 100 ml vodka (40% ethanol) providing a 4% solution, doses of which to be measured by means of a calibrated dropper. Initially a dose of 20 mg in 0.5 ml solution was added to fruit juice for convenience of administration. This 20 mg dose was given twice daily for 2 weeks, at which time the dose was increased to 40 mg in 1 ml solution twice daily for 3 weeks. Within the 5-week treatment period the patient showed signs of regaining short memory for events of the day. Recognition of family members and caregivers improved, as well as calling them by name. Physical activity increased. Sense of humor appeared and verbal conversation was attempted by the patient. Situational recognition returned to substantial degree. Singing of self-made up songs became a common daily event. Daytime urinary and bowel incontinence improved substantially.

The dose of N-acetyl-glutamic acid was then increased to 100 mg in 2.5 ml solution twice daily for 2 weeks. At this dose, the patient became verbally over-active, and would stay awake after dinner watching TV, resisting going to bed later in the evening. During the night, sleep talking and arm gesturing became excessive. N-Acetyl-glutamic acid treatment was then discontinued. Verbal over-activity diminished over the ensuing week.

The above result shows that N-acetylamino acids such as N-acetyl-glutamic acid is therapeutically effective for systemic treatment of nervous disorders such as Alzheimer's disease.

### EXAMPLE 20

The following is an example regarding synergetic or synergistic effects between pharmaceutical agents and the compounds useful in the embodiments. A male subject, age 71, having an itchy eruptive eczema all over the body, orally administered tetracycline hydrochloride 500 mg at 7:50 AM and oral prednisone 10 mg at 11:25 AM. The itch had subsided slightly. The subject then took oral diphenhydramine hydrochloride 25 mg at 4 PM and oral N-acetyl-L-prolinamide 200 mg at 9 PM. The itch stopped completely and the eruptive lesions of eczema subsided with substantial improvement over the next few days. The above result shows that pharmaceutical drugs can be orally administered with the compound of the present invention for synergetic or synergistic effects.

### EXAMPLE 21

The following is an example regarding synergetic or synergistic effects between vitamin, pharmaceutical agents and the substance of the present invention. A male subject, age 71, having an acute eczema lesions took oral tetracycline hydrochloride 500 mg at 8:30 AM and oral vitamin B5 (pantothenic acid calcium salt) 300 mg at 10:30 AM. Oral vitamin B5 300 mg was repeated at 3:30 PM. The subject took oral N-acetyl-DL-tryptophan 50 mg at 5 PM, 100 mg at 9 PM, and oral diphenhydramine hydrochloride 25 mg at 10 PM. The itch had stopped completely, and the acute eczema subsided with substantial improvement over the next few days.

The above result shows that vitamins and pharmaceutical drugs can be orally administered with the compound described in the embodiment herein for synergetic or synergistic effects.

### EXAMPLE 22

The following is another example regarding synergetic or synergistic effects of multiple pharmaceutical agents and the substance of the present invention. A male subject, age 71, having an acute nummular eczema took oral tetracycline hydrochloride 500 mg at 8:20 AM and oral N-acetyl-L-proline 100 mg at 10:30 AM and 200 mg at 2:30 PM. The subject took diphenhydramine hydrochloride 25 mg at 6:20 PM and tetracycline hydrochloride 500 mg at 9 PM. The itch had stopped completely, and the acute eczema subsided with substantial improvement over the next few days.

The above result shows that pharmaceutical drugs can be orally administered with the compound of the present invention for synergetic or synergistic effects.

What is claimed is:

1. A method of preventing or alleviating symptoms or syndromes associated with the nervous, vascular, muscoloskeletal, or cutaneous systems by sytemically administering to a mammal (human or animal) in need thereof, a therapeutically effective amount of a composition comprising at least one amino acid selected from the group consisting of alanine, glycine, isoleucine, proline, serine, valine, β-alanine, γ-aminobutanoic acid, citrulline and ornithine, present as a free acid, salt, partial salt, amide, lactone, ester, anhydride, dimer, oligomer or polymer form, D, L, or DL stereoisomers, non-stereoisomers, and mixtures thereof.

2. The method of claim 1, wherein the composition is in the form of a powder, solution, suspension, juice, tablet or gelatin capsule, and the amino acid is present in the composition in an amount with the range of from about 0.1 to 5 g.

3. The method of claim 1, wherein the composition is in the form of a solution or suspension for parenteral injection, and the amino acid is present in the solution or suspension in a concentration within the range of from about 1 to about 10%.

4. The method of claim 1, wherein the composition is systemically administered in an amount within the range of from about 1 to about 10 grams daily.

5. The method of claim 1, wherein the composition is systemically administered at least once daily.

6. The method of claim 5, wherein the composition is systemically administered at least twice daily.

7. The method of claim 1, wherein the amino acid is proline.

8. The method of claim 7, wherein the proline is present in a form selected from the group consisting of L-proline, sodium L-prolinate, L-prolinamide, ethyl L-prolinate, methyl L-prolinate, propyl L-prolinate, L-Pro-L-Pro dimer, (L-Pro-)₈ oligomer, ((L-Pro-)₂₀ polymer, D-proline, sodium D-prolinate, D-prolinamide, ethyl D-prolinate, methyl D-prolinate, propyl D-prolinate, DL-proline, sodium DL-prolinate, DL-prolinamide, ethyl DL-prolinate, methyl DL-prolinate, propyl DL-prolinate, and mixtures thereof.

9. The method of claim 1, wherein the amino acid is selected from the group consisting of L-proline, glycine, L-arginine, and mixtures thereof.

10. The method of claim 1, wherein the composition is systemically administered twice daily with different amino acids.

11. The method of claim 1, wherein the composition further comprises an additional agent selected from the group consisting of vitamins, cosmetics, pharmaceutical agents, and mixtures thereof.

12. A method of preventing or alleviating symptoms or syndromes associated with the nervous, vascular, muscoloskeletal, or cutaneous systems by sytemically administering to a mammal in need thereof, a therapeutically effective amount of a composition comprising at least one N-acetylamino acid selected from N-acetyl-proline or an N-acetyl amino acid represented by the formula:
R₁ CH(NHCOCH₃)(CH₂)ₙCOOR₂
where R₁, R₂ are independently H, an alkyl, aralkyl or aryl group having 1 to 14 carbon atoms; n is an integer ranging from 0 to 5, and R₁ may optionally be substituted with OH, SH, SCH₃, NH₂, CONH₂, NHCONH₂, NHC(═NH)NH₂, imidazole, pyrrolidine or other heterocyclic group, and wherein the H attached to any carbon atom can be substituted by I, F, Cl, Br, OH or alkoxy group having 1 to 9 carbons,
the N-acetylamino acids being present as free acid, salt, partial salt, amide, ester, anhydride, lactone form, D, L, or DL stereoisomers, non-stereoisomers, and mixtures thereof.

13. The method of claim 12, wherein the composition is in the form of a powder, solution, suspension, juice, tablet or gelatin capsule, and the N-acetyl proline or N-acetyl amino acid is present in the composition in an amount with the range of from about 20 to 500 mg.

14. The method of claim 12, wherein the composition is in the form of a solution or suspension for parenteral injection, and the N-acetyl proline or N-acetyl amino acid is present in the solution or suspension in a concentration within the range of from about 1 to about 10%.

15. The method of claim 12, wherein the composition is systemically administered in an amount within the range of from about 20 to about 500 mg daily.

16. The method of claim 12, wherein the composition is systemically administered at least once daily.

17. The method of claim 12, wherein the composition is systemically administered at least twice daily.

18. The method of claim 12, wherein the N-acetyl amino acid is selected from the group consisting of N-acetyl-alanine, N-acetyl-β-alanine, N-acetyl-γ-aminobutanoic acid, N-acetyl-β-aminoisobutanoic acid, N-acetyl-arginine, N-acetyl-asparagine, N-acetyl-aspartic acid, N-acetyl-citrulline, N-acetyl-dopa (N-acetyl-3,4-dihydroxyphenylalanine), N-acetyl-glycine, N-acetyl-glutamic acid, N-acetyl-glutamine, N-acetyl-histidine, N-acetyl-homoserine, N-acetyl-4-hydroxyproline, N-acetyl-isoleucine, N-acetyl-leucine, N-acetyl-lysine, N-acetyl-methionine, N-acetyl-ornithine, N-acetyl-phenylalanine, N-acetyl-proline, N-acetyl-serine, N-acetyl-threonine, N-acetyl-tryptophan, N-acetyl-tyrosine and N-acetyl-valine, and mixtures thereof.

19. The method of claim 12, wherein the N-acetyl amino acid is selected from the group consisting of N-acetyl-glutamic acid, N-acetyl-L-proline, N-acetyl-L-prolinamide, N-acetyl-L-glutamic acid, N-acetyl prolineethyl ester, N-acetyl-DL-tryptophan, and mixtures thereof.

20. The method of claim 12, wherein the composition further comprises an additional agent selected from the group consisting of vitamins, cosmetics, pharmaceutical agents, and mixtures thereof.

21. The method of claim 12, wherein the N-acetyl amino acid is N-acetyl glutamic acid, and the method alleviates symptoms associated with Alzheimer's disease.
